(19) United States
(12) Patent Application Publication
(10) Pub. No.: US 2026/0201308 A1
(43) Pub. Date: Jul. 16, 2026

(54) OBSERVATION CULTURE DEVICE

(71) Applicant: Yokogawa Electric Corporation, Musashino-shi, Tokyo (JP)

(72) Inventor: Yasunori YOKOYAMA, Musashino-shi (JP)

(73) Assignee: Yokogawa Electric Corporation, Musashino-shi, Tokyo (JP)

(21) Appl. No.: 19/157,133

(22) PCT Filed: Feb. 26, 2024
(86) PCT No.: PCT/JP2024/006778

§ 371 Date: Aug. 15, 2025

(30) Foreign Application Priority Data
Mar. 30, 2023 (JP) 2023-054781

(51) Int. Cl. C12M 1/34; C12M 1/00; C12M 1/12

(52) U.S. Cl. CPC C12M 41/36; C12M 25/10; C12M 29/18; C12M 41/14; C12M 41/34

(57) ABSTRACT

An object of the present disclosure is to provide an observation culture device which can stably maintain a culture space environment. An observation culture device (1) includes an observation container (10) which has an accommodation part having a sample accommodated therein; an observation device (20) which observes the accommodation part; an XY stage unit (30) which moves while supporting the observation container (10) so that the accommodation part faces the observation device (20); a top heater unit (40) and a bottom heater unit (50) disposed so that the XY stage unit (30) is disposed therebetween from the above and bottom to form a culture space (100) surrounding the observation container (10); and a humidifying unit (60) which supplies a humidified gas into the culture space (100). The humidifying unit (60) includes: a filling region filled with water for humidification; and a hollow fiber formed of a membrane through which water molecules pass and provided so that the membrane is in contact with the filling region, and a gas humidified using water molecules passing through the membrane of the hollow fiber is supplied into the culture space (100) as the humidified gas.

### TECHNICAL FIELD

The present disclosure relates to an observation culture device.

### BACKGROUND ART

Patent Literature 1 (PTL 1) which will be described below discloses a microscope observation culture device which allows microscopic observation to be performed while culturing living cells. This microscope observation culture device includes a top heater unit and a bottom heater unit in which an XY stage unit is disposed between the top heater unit and the bottom heater unit disposed from above and below. The XY stage unit includes a plate holder which holds a well plate having a sample being cultured accommodated therein. In addition, the bottom heater unit has one surface in which a flow path formed of a heater holder and a metal plate is formed and another surface in which a heater is provided.

### CITATION LIST

### Patent Literature

### [PTL 1]

Japanese Patent No. 6201647

### SUMMARY OF INVENTION

### Technical Problem

In the above technique in the related art, in order to perform microscopic observation while culturing living cells, the temperature, humidity, and CO₂ concentration of a culture space in which the well plate is placed are adjusted. To be specific, the humidity in the culture space is adjusted by supplying steam through micro-holes provided in the metal plate of the bottom heater unit. Furthermore, the CO₂ concentration in the culture space is adjusted by supplying a mixed gas including CO₂ through a CO₂ port provided in the plate holder.

However, if a mixed gas including CO₂ is supplied into the culture space, the humidity in the culture space decreases to that extent. Thus, maintaining a stable environment in the culture space is difficult.

For this reason, an object of the present disclosure is to provide an observation culture device which can maintain a stable environment in a culture space.

### Solution to Problem

In order to achieve the above object, an observation culture device according to a first aspect of the present disclosure includes: an observation container which has an accommodation part having a sample accommodated therein; an observation device which observes the accommodation part; an XY stage unit which moves while supporting the observation container so that the accommodation part faces the observation device; a top heater unit and a bottom heater unit disposed so that the XY stage unit is disposed therebetween from above and below to form a culture space surrounding the observation container; and a humidifying unit which supplies a humidified gas into the culture space, in which the humidifying unit includes: a filling region filled with water for humidification; and a hollow fiber formed of a membrane through which water molecules pass and provided so that the membrane is in contact with the filling region, and a gas humidified using water molecules passing through the membrane of the hollow fiber is supplied into the culture space as the humidified gas.

According to an observation culture device associated with a second aspect of the present disclosure, the observation culture device according to the first aspect of the present disclosure includes: a supply flow path which supplies the humidified gas from the humidifying unit into the culture space, in which the supply flow path is formed through a first through hole formed in a wall of the humidifying unit and a second through hole formed in a wall of the XY stage unit.

According to an observation culture device associated with a third aspect of the present disclosure, in the observation culture device according to the second aspect of the present disclosure, a wall of the humidifying unit and a wall of the XY stage unit are in contact with each other so that the first through hole and the second through hole communicate with each other.

According to an observation culture device associated with a fourth aspect of the present disclosure, in the observation culture device according to the third aspect of the present disclosure, the XY stage unit includes a heater unit, and the humidifying unit is heated through heat conduction from the wall of the XY stage unit heated using the heater unit.

According to an observation culture device associated with a fifth aspect of the present disclosure, in the observation culture device according to any one of the second aspect to the fourth aspect of the present disclosure, the filling region is outside of the hollow fiber, and an inside of the hollow fiber communicates with the supply flow path.

According to an observation culture device associated with a sixth aspect of the present disclosure, in the observation culture device according to any one of the first aspect to the fifth aspect of the present disclosure, the humidifying unit is disposed in a region which is located between the top heater unit and the bottom heater unit.

According to an observation culture device associated with a seventh aspect of the present disclosure, the observation culture device according to any one of the first aspect to the sixth aspect of the present disclosure includes a water supply flow path connected to the humidifying unit and configured to supply water for humidification to the filling region.

### Advantageous Effects of Invention

According to an aspect of the present disclosure, an observation culture device which can stably maintain an environment in a culture space can be obtained.

### BRIEF DESCRIPTION OF DRAWINGS

FIG. 1 is a constitution diagram of an observation culture device 1 according to an embodiment of the present disclosure.

FIG. 2 is a view taken along an arrow A shown in FIG. 1.

FIG. 3 is a view taken along an arrow B shown in FIG. 1.

FIG. 4 is a view taken along an arrow C shown in FIG. 1.

FIG. 5 is a view taken along an arrow D shown in FIG. 1.

FIG. 6 is a diagram showing a situation when an objective lens according to the embodiment of the present disclosure is raised.

FIG. 7 is an internal constitution diagram of a humidifying unit according to the embodiment of the present disclosure.

### DESCRIPTION OF EMBODIMENTS

An observation culture device according to embodiments of the present disclosure will be described in detail below with reference to the drawings. First, an overview of the embodiments of the present disclosure will be described below, and then the details of each of the embodiments of the present disclosure will be described.

### Overview

In the constitution described in Patent Literature 1 described above, a means for providing moisture to a culture space and a means for supplying a mixed gas containing CO₂ to the culture space are separately provided.

With regard to CO₂, a CO₂ concentration in the culture space is set to 5% by supplying a mixed gas whose CO₂ concentration is adjusted to, for example, 5% from the outside through a CO₂ port (not shown) of a plate holder. Furthermore, with regard to humidity, water is evaporated through a plurality of micro-holes provided in a metal plate of a bottom heater unit, thereby forming a high humidity environment in the culture space.

Incidentally, in order to stabilize the CO₂ concentration in the culture space, increasing a flow rate of a mixed gas supplied from the outside of the culture space is required. However, the mixed gas is not humidified in the constitution in the related art described above. Thus, if the flow rate of the mixed gas is increased, the humidity in the culture space decreases.

Raising a temperature of the bottom heater unit which supplies steam is required to increase the decreased humidity. Here, if this happens, a temperature distribution in the culture space collapses. In this way, in the above-described technique in the related art, in order to maintain a stable environment in the culture space, setting the temperature of each heater unit, the CO₂ concentration of the mixed gas, and the flow rate very strictly is required.

In the embodiment of the present disclosure, a pre-humidified gas (humidified gas) is supplied into the culture space using a humidifying unit including hollow fibers. Since each of the hollow fibers is formed of a membrane through which water molecules pass and is in contact with a filling region filled with water over a wide area, the mixed gas supplied to the culture space can be sufficiently humidified. Thus, the dependence of temperature control with respect to humidification is reduced compared to the above-described technique in the related art, thereby facilitating temperature control of the culture space.

Therefore, a stable culture environment can be formed without requiring the strict setting of the temperature of the heater unit, the CO₂ concentration of the mixed gas, and the flow rate.

### Embodiment

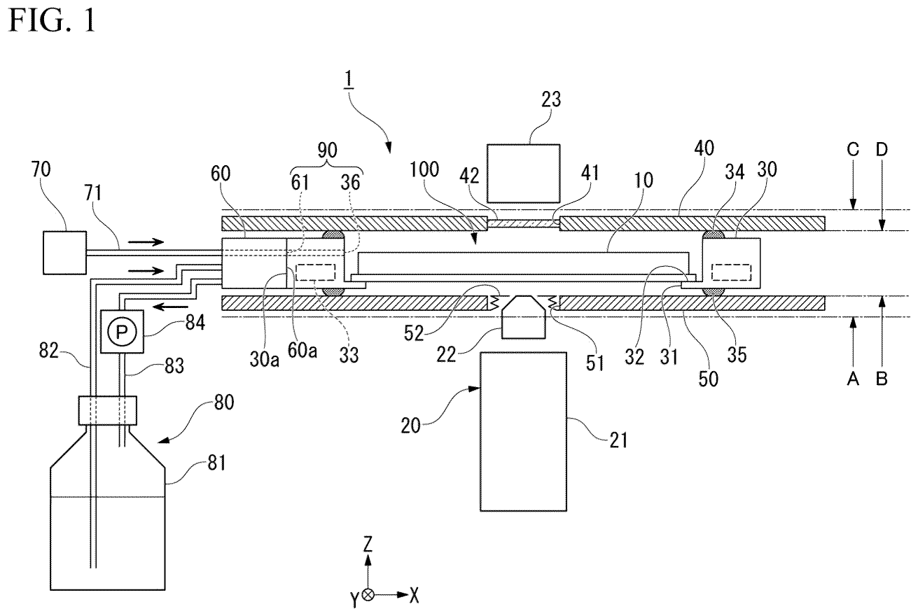

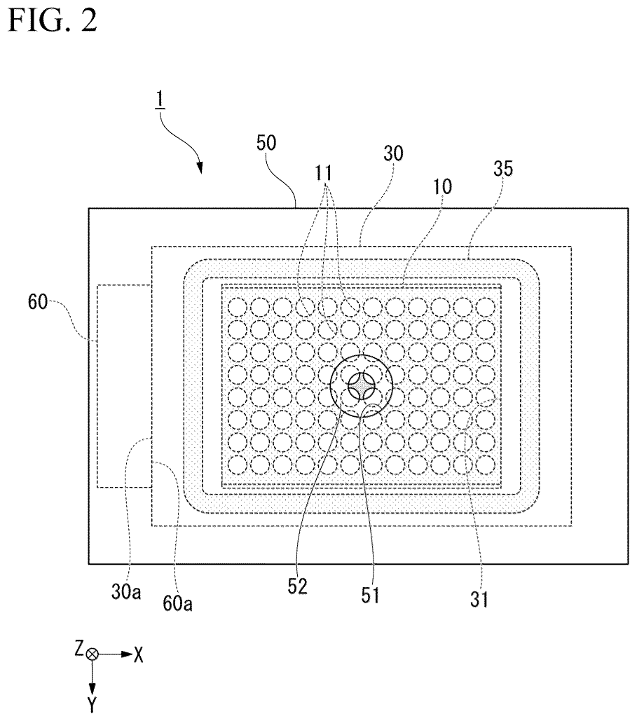

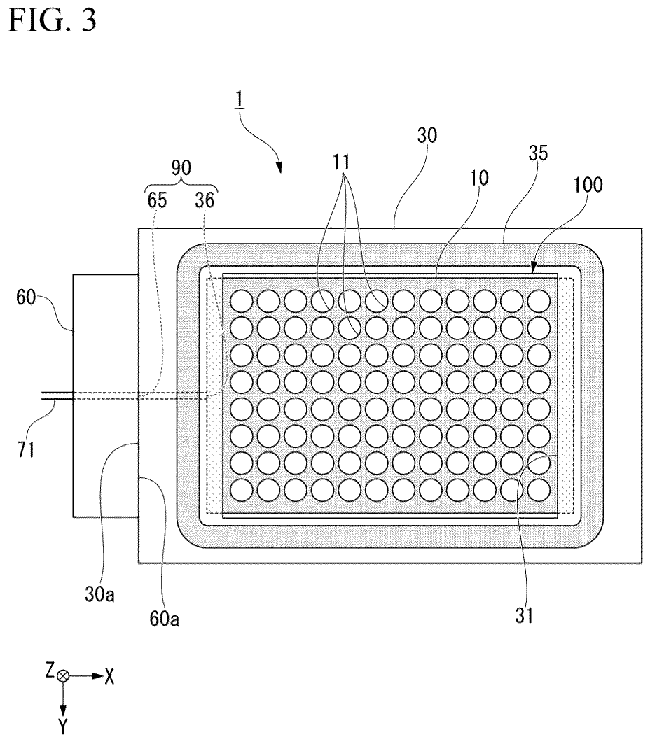

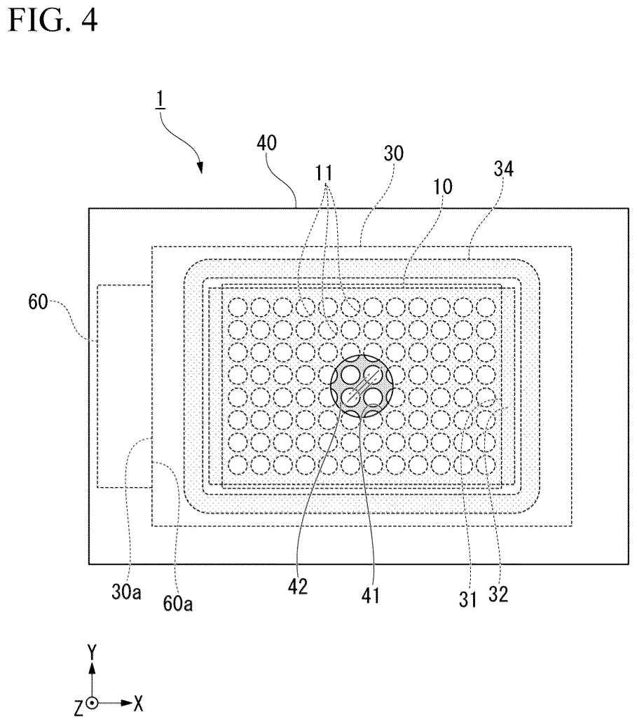

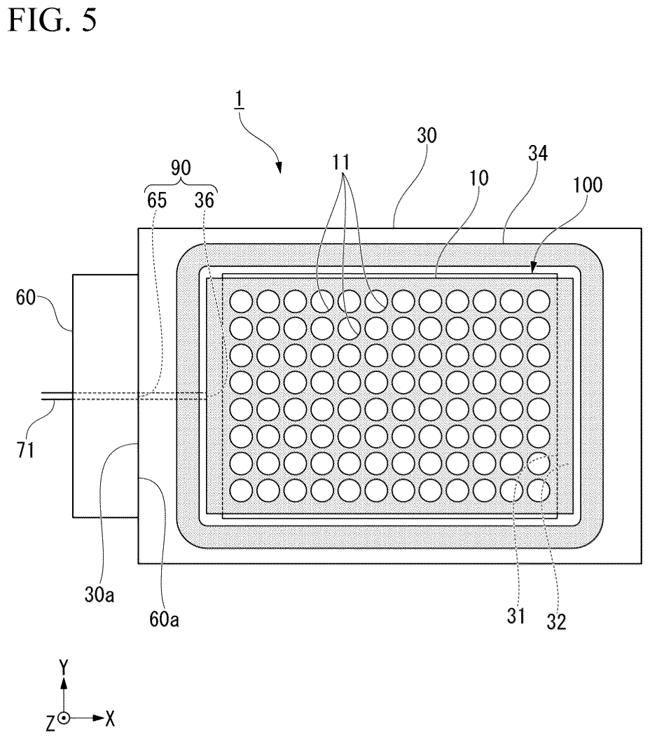

FIG. 1 is a constitution diagram of an observation culture device 1 according to an embodiment of the present disclosure. FIG. 2 is a view taken along an arrow A shown in FIG. 1. FIG. 3 is a view taken along an arrow B shown in FIG. 1. FIG. 4 is a view taken along an arrow C shown in FIG. 1. FIG. 5 is a view taken along an arrow D shown in FIG. 1.

As shown in FIG. 1, the observation culture device 1 includes an observation container 10, an observation device 20, an XY stage unit 30, a top heater unit 40, a bottom heater unit 50, and a humidifying unit 60.

In the following description, an XYZ orthogonal coordinate system may be set and a positional relationship of members may be explained with reference to the XYZ orthogonal coordinate system in some cases. An X-axis direction and a Y-axis direction are biaxial orthogonal directions (horizontal direction) which are orthogonal to each other in the horizontal plane. A Z-axis direction is a vertical direction which is orthogonal to the X-axis direction and the Y-axis direction.

As shown in FIG. 5, the observation container 10 has accommodation parts 11 having a sample such as cells accommodated therein. The observation container 10 in the embodiment is a multi-well plate having the plurality of accommodation parts 11 (wells). The number of accommodation parts 11 may be one. The observation container 10 is formed into a rectangular shape in a plan view. The entire observation container 10 or at least a bottom part of the accommodation part 11 may be made of a transparent material.

As shown in FIG. 1, the observation device 20 observes the accommodation part 11 from a bottom surface side of the observation container 10. The observation device 20 in the embodiment includes an imaging device 21, an objective lens 22, and an illumination device 23. The imaging device 21 and the objective lens 22 are disposed on a side below (−Z side of) the observation container 10. The illumination device 23 is disposed above the observation container 10 (+Z side).

The imaging device 21 images the inside of the accommodation part 11 via the objective lens 22. The objective lens 22 is constituted to be movable in the Z-axis direction using an actuator (not shown). The illumination device 23 illuminates the inside of the accommodation part 11 through a window part 42 provided in the top heater unit 40. The observation device 20 in the embodiment has a constitution of an inverted microscope, but may have a constitution of an upright microscope.

The XY stage unit 30 supports the observation container 10 and moves along the XY plane so that the accommodation part 11 and the observation device 20 face each other. The XY stage unit 30 is formed into a rectangular frame shape in a plan view and has an opening portion 31 having the observation container 10 accommodated therein. A support portion 32 which supports the observation container 10 is formed on a circumferential edge portion of an opening the opening portion 31.

A heater unit 33 which heats the observation container 10 to a predetermined temperature (for example, about 37° C.) is provided inside the XY stage unit 30. An annular sealing material 34 (refer to FIG. 5) configured to maintain airtightness between the XY stage unit 30 and the top heater unit 40 is provided on an upper surface of the XY stage unit 30. Furthermore, an annular sealing material 35 (see FIG. 3) configured to maintain airtightness between the XY stage unit 30 and the bottom heater unit 50 is provided on a lower surface of the XY stage unit 30.

The XY stage unit 30 shown in FIG. 1 is configured to be able to move the observation container 10 in the X-axis direction and the Y-axis direction using the actuator (not shown). Although it is preferable that the sealing material 34 and the top heater unit 40 and the sealing material 35 and the bottom heater unit 50 be in contact with each other, when an operation of the XY stage unit 30 is affected, a slight gap may be allowed as long as sufficient airtightness is maintained.

For example, the XY stage unit 30 is configured to perform large driving in at least one of the X-axis direction and the Y-axis direction so that the observation container 10 can be moved outside of a position covered by the top heater unit 40 and the observation container 10 can be attached and detached. A through hole 36 which passes through a wall 30a in the horizontal direction is formed the wall 30a of the XY stage unit 30. The through hole 36 forms a supply flow path 90 for a humidified gas which will be described later.

The top heater unit 40 is disposed above the XY stage unit 30 (+Z side). As shown in FIG. 4, the top heater unit 40 is formed into a rectangular plate shape in a plan view. An opening portion 41 is formed in a center of the top heater unit 40. The window part 42 made of a transparent material is fitted into the opening portion 41 so that the illumination light from the illumination device 23 (refer to FIG. 1) can pass therethrough. It is preferable that the window part 42 be a transparent panel heater to prevent condensation.

As shown in FIG. 1, the bottom heater unit 50 is disposed below the XY stage unit 30 (−Z side). Furthermore, as shown in FIG. 2, the bottom heater unit 50 is formed into a rectangular plate shape when viewed from the bottom. An opening portion 51 is formed in a center of the bottom heater unit 50. A bellows 52 is attached to the opening portion 51 and can expand and contract in the Z-axis direction together with the objective lens 22.

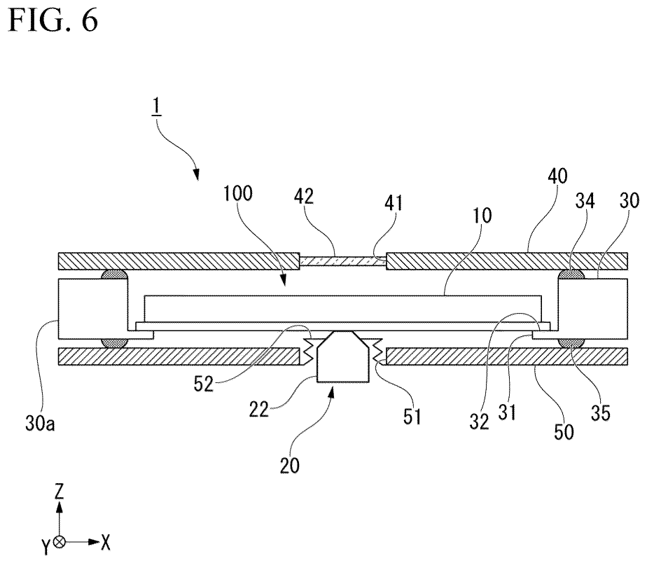

FIG. 6 is a diagram showing a situation when the objective lens 22 according to the embodiment of the present disclosure is raised.

As shown in FIG. 6, a constitution in which, when the objective lens 22 is in a raised position, the objective lens 22 pushes up the bellows 52 and the opening portion 51 of the bottom heater unit 50 is sealed using the objective lens 22 and the bellows 52 is provided.

The top heater unit 40 and the bottom heater unit 50 are disposed so that the XY stage unit 30 is disposed therebetween from above and below and form a culture space 100 surrounding the observation container 10. The culture space 100 corresponds to an environment appropriate for culturing a sample and, for example, the humidity, temperature, CO₂ concentration, and the like therein are adjusted.

The top heater unit 40 and the bottom heater unit 50 heat the culture space 100 to a predetermined temperature (for example, about 37° C.). The objective lens 22 inserted into the culture space 100 may also include a heater configured to stabilize a temperature of the culture space 100.

As shown in FIG. 1, the humidifying unit 60 is attached to the outside of the XY stage unit 30 and supplies a humidified gas into the culture space 100. A size of the humidifying unit 60 can be designed independently of the XY stage unit 30 and the observation container 10. For example, the humidifying unit 60 can be made smaller without depending on a size of the observation container 10. The humidifying unit 60 is disposed in a region which is located between the top heater unit 40 and the bottom heater unit 50.

An air supply unit 70 and a water supply unit 80 are connected to the humidifying unit 60. The air supply unit 70 is connected to the humidifying unit 60 via an air supply flow path 71. The air supply unit 70 generates a mixed gas with a CO₂ concentration adjusted to, for example, 5%. The air supply unit 70 may include a gas cylinder containing a mixed gas whose component has been adjusted in advance.

The water supply unit 80 includes a bottle 81 having water for humidification accommodated therein and a water supply flow path 82 and a water discharge flow path 83 (drainage flow path) which connect the bottle 81 and the humidifying unit 60. One end of the water supply flow path 82 is disposed in a liquid inside the bottle 81. The other end of the water supply flow path 82 is connected to the humidifying unit 60.

One end of the water discharge flow path 83 is connected to the humidifying unit 60. The other end of the water discharge flow path 83 is disposed in the air inside the bottle 81. A pump 84 is provided in the water discharge flow path 83. If the pump 84 is driven, water inside the bottle 81 is suctioned up from one end of the water supply flow path 82 and supplied from the other end of the water supply flow path 82 to the humidifying unit 60. Furthermore, water inside the humidifying unit 60 is suctioned out from one end of the water discharge flow path 83 and discharged from the other end of the water discharge flow path 83 to the bottle 81.

The pump 84 may be a syringe pump, a peristaltic pump, a bellows pump, or the like. Furthermore, the pump 84 may be provided in the water supply flow path 82 or in both the water supply flow path 82 and the water discharge flow path 83. Furthermore, although one bottle 81 is shown for water supply and discharge, it is also possible to have separate bottles for water supply and discharge or there may be a tank.

A humidified gas generated in the humidifying unit 60 is supplied from the humidifying unit 60 to the culture space 100 via the supply flow path 90. The supply flow path 90 is formed through a through hole 61 formed in a wall 60a of the humidifying unit 60 and the through hole 36 formed in the wall 30a of the XY stage unit 30.

In the embodiment, the wall 60a of the humidifying unit 60 and the wall 30a of the XY stage unit 30 are in contact with each other so that the through hole 61 and the through hole 36 communicate with each other. That is to say, the humidifying unit 60 is sufficiently thermally connected to the XY stage unit 30 and is configured so that a temperature thereof can be controlled using the heater unit 33 attached to the XY stage unit 30.

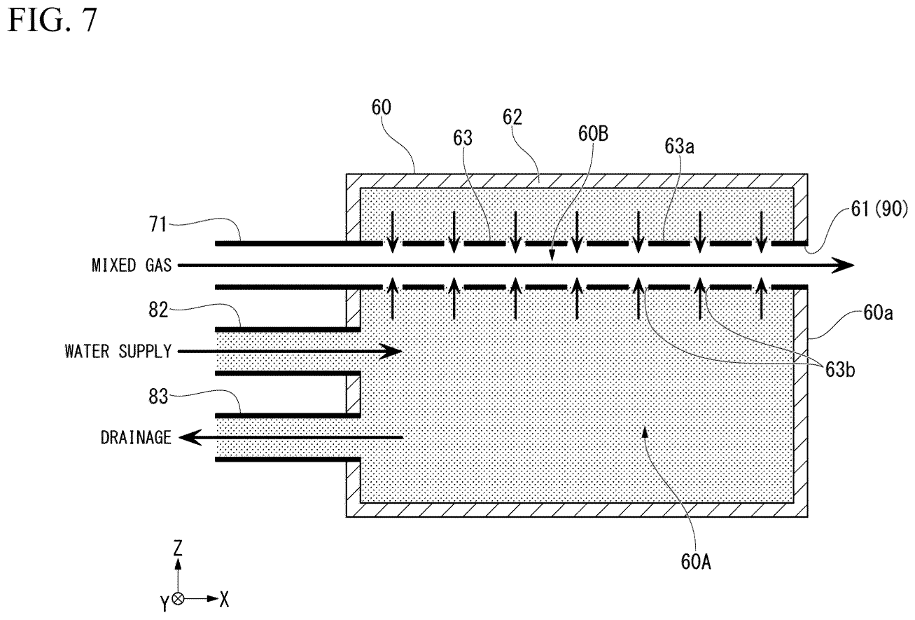

FIG. 7 is an internal constitution diagram of the humidifying unit 60 according to the embodiment of the present disclosure.

As shown in FIG. 7, the humidifying unit 60 includes a case 62 and hollow fiber 63. The case 62 of the embodiment is formed into a rectangular box shape. The case 62 is connected to the air supply flow path 71, the water supply flow path 82, and the water discharge flow path 83 described above. Furthermore, the through hole 61 which forms the supply flow path 90 for a humidified gas is formed in the wall 60a of the case 62.

The hollow fiber 63 is disposed inside the case 62 and has one end connected to the air supply flow path 71 and the other end connected to the through hole 61 (supply flow path 90). Although the hollow fiber 63 is schematically illustrated in FIG. 7, there may be a plurality of the hollow fibers 63 or the hollow fibers 63 may be in a bundle. Furthermore, the hollow fiber 63 may not only extend linearly inside the case 62, but may also be curved or meandering.

The case 62 forms a filling region 60A filled with water for humidification. The water supply flow path 82 and the water discharge flow path 83 are connected to the filling region 60A. The hollow fiber 63 is composed of a cylindrical membrane 63a and a hollow portion 60B that is an internal space thereof. The membrane 63a is in contact with the filling region 60A. A plurality of micro-holes 63b are formed in the membrane 63a. Although liquid water cannot pass through the micro-holes 63b, gaseous water molecules can pass therethrough. Such a membrane 63a is also called a semi-transparent membrane or the like.

An operation of the observation culture device 1 having the above constitution will be explained below.

First, the XY stage unit 30 shown in FIG. 1 is moved outside of a position covered by the top heater unit 40 and the observation container 10 is placed on the XY stage unit 30 by a user, a robot, or the like.

Subsequently, the XY stage unit 30 moves the observation container 10 between the top heater unit 40 and the bottom heater unit 50 and raises the objective lens 22. Thus, the culture space 100 in which the observation container 10 is disposed becomes airtight from the outside.

Subsequently, the temperature of the culture space 100 is maintained at conditions appropriate for culturing a sample such as cells (for example, about 37° C.) by controlling the temperatures of the top heater unit 40, the bottom heater unit 50, and the heater unit 33 attached to the XY stage unit 30. A culture state of the sample can be maintained even better by performing the above temperature control in advance before installing the observation container 10.

The pump 84 supplies the water in the bottle 81 to the filling region 60A of the humidifying unit 60 via the water supply flow path 82. On the other hand, the pump 84 discharges excess water from the filling region 60A to the bottle 81 via the water discharge flow path 83. Since the humidifying unit 60 is installed outside of the XY stage unit 30, the impact on the environment of the culture space 100 (temperature change, humidity change, and the like) due to water supply to the filling region 60A and water discharge from the filling region 60A is extremely small.

A mixed gas whose component has been adjusted using the air supply unit 70 is supplied to the culture space 100 via the hollow fiber 63 and the supply flow path 90 inside the humidifying unit 60. As shown in FIG. 7, the mixed gas is humidified when passing through the hollow portion 60B of the hollow fiber 63. The membrane 63a of the hollow fiber 63 has the plurality of micro-holes 63b. Although liquid water cannot pass through the micro-holes 63b, gaseous water molecules can pass through the micro-holes 63b.

That is to say, the water molecules enter the hollow portion 60B from the filling region 60A via the micro-holes 63b and humidify the mixed gas passing through the hollow portion 60B. Furthermore, since liquid water does not enter the hollow portion 60B of the hollow fiber 63 from the filling region 60A, the risk of water leakage into the culture space 100 can be reduced.

Even if bacteria or the like grow in the filling region 60A, they cannot pass through the micro-holes 63b. Since such bacteria are not included in the mixed gas, they do not mix with the sample in the observation container 10 and the risk of contamination can be reduced.

The larger the surface area of the hollow fiber 63 (area of the membrane 63a), the easier it is to obtain a humidifying function. Since the hollow fiber 63 is a thread and has a small cross-sectional area, the ratio of surface area to volume of the hollow fiber 63 is extremely large. A sufficient humidification function can be obtained even in a small volume using the hollow fiber 63.

Referring to FIG. 1 again, at the time of imaging the sample cultured in the observation container 10, the observation container 10 is moved along the XY plane using the XY stage unit 30 and the accommodation part 11 and the observation device 20 are made to face each other. Furthermore, the objective lens 22 is moved in the Z-axis direction and moved to a position in which it is in focus with the accommodation part 11.

Also, the sample is illuminated using transmitted illumination through the illumination device 23 or epi-illumination (not shown) via the objective lens 22 and the sample is imaged using the imaging device 21.

The movement of the XY stage unit 30, the movement of the objective lens 22, the temperature control of the top heater unit 40, the bottom heater unit 50, and the heater unit 33 attached to the XY stage unit 30, the gas supply of the air supply unit 70, the water supply/discharge using the pump 84, operations of the illumination device 23 or epi-illumination (not shown), and the operation using the imaging device 21 which have been described above may be automatically performed using a controller (not shown).

In the observation culture device 1 having the above constitution, the humidifying unit including the hollow fiber 63 supplies a pre-humidified mixed gas (humidified gas) into the culture space. The gas components (for example, CO₂ concentration) and humidity of the culture space 100 are stabilized by supplying the mixed gas to the culture space 100 in a sufficiently humidified state using the humidifying unit 60.

The hollow fiber 63 is formed using the membrane 63a through which water molecules are allowed to pass and has a large area in which it is in contact with the filling region 60A which is filled with water. Thus, the mixed gas supplied into the culture space 100 can be sufficiently humidified. Therefore, since the dependence of temperature control on humidification is reduced compared to the method in the related art, temperature control of the culture space 100 becomes easier.

The above two points make it possible to form the stable culture space 100 which does not require strict heater temperature settings, or a mixed gas CO₂ concentration, and flow rate settings.

In this way, the observation culture device 1 according to the embodiment includes the observation container 10 which has the accommodation part 11 having a sample accommodated therein, the observation device 20 which observes the accommodation part 11, the XY stage unit 30 which moves while supporting the observation container 10 so that the accommodation part 11 faces the observation device 20, the top heater unit 40 and the bottom heater unit 50 disposed so that the XY stage unit 30 is disposed therebetween from the above and bottom to form the culture space 100 surrounding the observation container 10, and the humidifying unit 60 which supplies a humidified gas into the culture space 100. In addition, the humidifying unit 60 includes the filling region 60A filled with water for humidification and the hollow fiber 63 formed of the membrane 63a through which water molecules pass and provided so that the membrane 63a is in contact with the filling region 60A. Moreover, a gas humidified using water molecules passing through the membrane 63a of the hollow fiber 63 is supplied into the culture space 100 as the humidified gas. According to this constitution, the stable culture space 100 can be formed without requiring strict heater unit temperature settings, a mixed gas CO₂ concentration, and flow rate settings.

Also, the embodiment includes the supply flow path 90 which supplies a humidified gas from the humidifying unit 60 to the culture space 100. In addition, the supply flow path 90 is formed through the through hole 61 (first through hole) formed in the wall 60a of the humidifying unit 60 and the through hole 36 (second through hole) formed in the wall 30a of the XY stage unit 30. According to this constitution, the humidified gas can be supplied from the humidifying unit 60 located outside of the XY stage unit 30 to the culture space 100 via the supply flow path 90.

Furthermore, in the embodiment, the wall 60a of the humidifying unit 60 and the wall 30a of the XY stage unit 30 are in contact with each other so that the through hole 61 and the through hole 36 communicate with each other. According to this constitution, the humidified gas can be supplied into the culture space 100 over the shortest distance. Moreover, when the XY stage unit 30 and the humidifying unit 60 are disposed together, the observation culture device 1 can be made smaller.

In addition, in the embodiment, the XY stage unit 30 includes the heater unit 33 and the humidifying unit 60 is heated through heat conduction from the wall 30a of the XY stage unit 30 heated using the heater unit 33. According to this constitution, the temperature of the humidifying unit 60 can be controlled using the heater unit 33 attached to the XY stage unit 30.

Moreover, in the embodiment, the humidifying unit 60 is disposed in a region which is located between the top heater unit 40 and the bottom heater unit 50. According to this constitution, the top heater unit 40 and the bottom heater unit 50 can further control the temperature of the humidifying unit 60.

Also, the embodiment includes the water supply flow path 82 connected to the humidifying unit 60 and configured to supply water for humidification to the filling region 60A. According to this constitution, the filling region 60A can be replenished with water consumed by humidifying the mixed gas.

Furthermore, in the embodiment, the filling region 60A is outside of the hollow fiber 63 and the inside of the hollow fiber 63 communicates with the supply flow path 90. According to this constitution, supplying water to the outside of the hollow fiber 63 rather than supplying water to the inside of the hollow fiber 63 can reduce the load (power consumption due to flow path resistance) on the pump 84.

Although the preferred embodiment of the present disclosure has been described above with reference to the drawings, the present disclosure is not limited to the embodiment described above. The shapes, the combinations, and the like of each constituent member shown in the embodiment described above are merely examples and various changes can be provided on the basis of design requirements and the like without departing from the spirit of the present disclosure.

### REFERENCE SIGNS LIST

1 Observation culture device
10 Observation container
11 Accommodation part
20 Observation device
21 Imaging device
22 Objective lens
23 Illumination device
30 XY stage unit
30a Wall
31 Opening portion
32 Support part
33 Heater unit
34 Sealing material
35 Sealing material
36 Through hole (second through hole)
40 Top heater unit
41 Opening portion
42 Window part
50 Bottom heater unit
51 Opening portion
52 Bellows
60 Humidifying unit
60a Wall
60A Filling region
60B Hollow portion
61 Through hole (first through hole)
62 Case
63 Hollow fiber
63a Membrane
63b Micro-hole
70 Air supply unit
71 Air supply flow path
80 Water supply unit
81 Bottle
82 Water supply flow path
83 Water discharge flow path
84 Pump
90 Supply flow path
100 Culture space

1. An observation culture device comprising:
an observation container which has an accommodation part having a sample accommodated therein;
an observation device which observes the accommodation part;
an XY stage unit which moves while supporting the observation container so that the accommodation part faces the observation device;
a top heater unit and a bottom heater unit disposed so that the XY stage unit is disposed therebetween from the above and bottom to form a culture space surrounding the observation container; and
a humidifying unit which supplies a humidified gas into the culture space,
wherein the humidifying unit includes:
a filling region filled with water for humidification; and
a hollow fiber formed of a membrane through which water molecules pass and provided so that the membrane is in contact with the filling region, and
a gas humidified using water molecules passing through the membrane of the hollow fiber is supplied into the culture space as the humidified gas.

2. The observation culture device according to claim 1, comprising:
a supply flow path which supplies the humidified gas from the humidifying unit into the culture space,
wherein the supply flow path is formed through a first through hole formed in a wall of the humidifying unit and a second through hole formed in a wall of the XY stage unit.

3. The observation culture device according to claim 2, wherein a wall of the humidifying unit and a wall of the XY stage unit are in contact with each other so that the first through hole and the second through hole communicate with each other.

4. The observation culture device according to claim 3, wherein the XY stage unit includes a heater unit, and
the humidifying unit is heated through heat conduction from the wall of the XY stage unit heated using the heater unit.

5. The observation culture device according to claim 2, wherein the filling region is outside of the hollow fiber, and
an inside of the hollow fiber communicates with the supply flow path.

6. The observation culture device according to claim 1, wherein the humidifying unit is disposed in a region which is located between the top heater unit and the bottom heater unit.

7. The observation culture device according to claim 1, comprising:
a water supply flow path connected to the humidifying unit and configured to supply water for humidification to the filling region.
